(19) United States
(12) Patent Application Publication
(10) Pub. No.: US 2004/0132175 A1
(43) Pub. Date: Jul. 8, 2004

(54) Cell culture chamber and bioreactor for extracorporeal culture of animal cells

(76) Inventors: Jerome Vetillard, Lognes (FR); Francis Herodin, Crolles (FR); Richard Caterini, Orlienas (FR)

Correspondence Address: MCCARTER & ENGLISH LLP, CITYPLACE I, 185 ASYLUM STREET, HARTFORD, CT, 06103, US

(21) Appl. No.: 10/333,377

(22) PCT Filed: Jul. 19, 2001
(86) PCT No.: PCT/FR01/02358

(30) Foreign Application Priority Data
Jul. 19, 2000 (EP) 00115568.8

(51) Int. Cl. C12M 1/12

(52) U.S. Cl. 435/297.1

(57) ABSTRACT

The invention concerns a cell culture chamber having at least two planar filtering membranes with different cut-off, delimited by an envelope with axis of symmetry formed by an outer lateral wall, two end walls and inlet ports and outlet ports for dynamic liquid media, and a bioreactor containing the culture chamber for extracorporeal culture of animal cells.

### FIELD OF THE INVENTION

The present invention concerns a cell culture chamber and a bioreactor containing the culture chamber for the extracorporeal culture of animal cells.

The invention relates more particularly to a cell culture chamber having at least two filtering planar membranes with different cutting threshold, delimited by an envelope with axis of symmetry and a bioreactor which allows the culture of animal cells in said culture chamber while regulating and controlling the environment in which the cells are cultivated.

The cell culture chamber and the bioreactor of the invention for extracorporeal culture of animal cells allow the culture of animal cells under sterile conditions.

The invention applies to the production of animal cells such as, for example, the hematopoietic cells, the hepatic cells, the cells of the skin (called keratinocytes), the cells of pancreas, the nervous cells organized or not in tissue structure with a therapeutic aim.

### STATE OF THE ART

The transplantation of organs, of tissues or of cells representing a considerable medical technological potential, a significant economic interest has consequently developed for the realization of bioreactors intended for the culture of animal cells with a therapeutic aim, either by grafting said cells, or by using them in external devices interfaced with the patient as palliatives means in case of organic deficiency.

A major disadvantage of the current bioreactors intended for the culture of eucaryotes cells lies in the mass transfer, that is to say the mass transfer of nutriments and of dissolved oxygen up to the eucaryotes cells, because these cells are fragile and are destroyed by the mechanical stress generated by the stirring of the media in order to ventilate the latter.

The American U.S. Pat. No. 6,048,721 describes a bioreactor for the ex vivo growth and maintenance of mammalian cells. In this bioreactor, the substantially circular culture chamber is delimited by a planar bed of cells and a gas permeable, liquid impervious membrane. The nutrient media injected in the lower compartment of the bioreactor diffuses radially and the air insufflated in the higher compartment oxygenates the media. According to the operating mode of this bioreactor, the media charged with the waste generated by the culture of the cells is disposed of. The recovery of the cells after culture is done according to an enzymatic processing. In this type of device, the thickness of the culture chamber may induce a gradient of an oxygen partial pressure which is little adapted to a good cell viability.

The drawback of bioreactors known by the prior art and intended for the culture of eucaryote cells lies in the fact that these bioreactors function in continuous perfusion of their culture chamber, the flow of the nutrient media is not recovered and is directly disposed of, increasing in a significant way the cost of the culture.

However, for the culture of animal cells, such as for example the hematopoietic cells or the hepatic cells, a nutrient flow rich in growth factors appears absolutely necessary to allow the multiplication and differentiation of said cells. Said growth factors being extremely expensive, the operation of a bioreactor, which does not permit the recycling of these factors, represents a considerable financial cost which is incompatible with a clinical exploitation. The purpose of the present invention is to avoid all these drawbacks.

### OBJECT OF THE INVENTION

The aim of the present invention is to create a culture chamber and a bioreactor for extracorporeal culture of animal cells, making it possible to preserve the homeostasy of the media surrounding the cultivated cells and thus to enable them to proliferate under the best possible conditions.

In order to achieve this aim, the invention develops the objectives hereafter exposed.

An object of the invention is to maintain a good cellular viability within said culture chamber and bioreactor, and this, by providing, on the one hand to the cells of the culture media a nutriment supply in an adequate amount and, on the other hand by evacuating the waste and the inhibitor elements generated in order to allow a growth of the cell population.

An other object of the invention is to be able to recycle the growth factors of the media while evacuating from the culture media, sufficiently cell wastes and thus to achieve the economic optimization of the cells culture.

Another object of the invention is to be able to carry out a transfer of gene targeted onto the cultivated cells.

Another object of the invention is to maintain the physico-chemical properties of the cell culture media, in spite of the disturbance induced by the cell growth.

Another object of this invention is to guarantee the sterility and asepsis of the culture chamber and the bioreactor, and in particular throughout the entire cell culture process.

Another object of the invention is to recover the cells cultivated within the culture chamber and the bioreactor of the invention.

### SUMMARY OF THE INVENTION

The present invention is based on the observation wherein a culture chamber and a bioreactor for extracorporeal culture of animal cells could remedy to the different drawbacks mentioned above if they allowed at the same time the maintaining of a good cellular viability of the cells cultivated, while allowing the recycling of the growth factors of the media, thus ensuring a good level of cellular proliferation.

Thus, the invention has for object a culture chamber for extracorporeal culture of animal cells, delimited by an envelope with axis of symmetry, which is formed of an external lateral wall, of two end walls and of inlets and outlets of the dynamic liquid media, said chamber being characterized in that it comprises:

a) at least two filtering planar membranes with different cutting threshold, perpendicular to the axis of symmetry;

b) between the membranes, a means forming a biocompatible culture support allowing the adhesion of cells in the state of culture;

c) two end walls constituting means of distribution of the dynamic liquid media;

d) three inlet and outlet couples of the dynamic liquid media (F1, F2, F3), intended to feed the cells culture chamber and to selectively extract the cultivated cells, the waste resulting from their culture and the nutrients in excess, two of the couples being, for each of them, connected between one of the end walls and one of the membranes, the third couple being connected between the two filtering planar membranes.

The invention also has for object a bioreactor for extracorporeal culture of animal cells comprising a culture chamber, delimited by an envelope with axis of symmetry, said envelope being formed of an external lateral wall, two ends walls and of inlets and outlets of the dynamic liquid media, and comprising means of circulation of said media in said chamber, this bioreactor being characterized in that it comprises:

a) a culture chamber of said cells comprising at least two filtering planar membranes with different cutting threshold, positioned perpendicularly to the axis of symmetry, and between said membranes with different cutting threshold a means forming a biocompatible culture support is located, this means allowing the adhesion of the growing cells, said chamber being delimited by an external envelope with axis of symmetry comprising two end walls constituting means of distribution for the circulation of the dynamic liquid media, and three inlet and outlet couples of the dynamic liquid media F1, F2, F3 intended to feed the cells culture chamber and to selectively extract the cultivated cells, the waste resulting from their culture and the excess nutrients, two of the coupled devices being, for each of them, connected between one of the end walls and one of the membranes, the third one being connected between the two filtering membranes;

b) circulation means for the first dynamic liquid media F1 operating in a closed loop and an expansion vessel R1 containing said media, said means being connected to said culture chamber;

c) circulation means for the second dynamic liquid media F2 operating in a closed loop or in an open loop, depending upon the intended function dedicated to said media, and a tank R2 containing said media, these means being connected to said culture chamber;

d) circulation generating means for the third dynamic liquid media F3 operating in an open loop and a tank R3 containing said media, these means being connected to said culture chamber;

e) control, regulation and conditioning means for the dynamic liquid media linked to a regulation-control unit.

### BRIEF DESCRIPTION OF THE DRAWINGS

The invention will be better understood with the non-restrictive illustrative description of the culture chamber and the bioreactor comprising said chamber, by means of the figures presented thereafter:

FIG. 1 represents a schematic view of a cell culture chamber delimited by an envelope with an axis of symmetry of hexagonal shape.

FIG. 2 schematically represents the dynamic circulation of liquid media F1 and F3 for a gradient of pressure p1>p3 in a “downward phase” within the culture chamber of a bioreactor of the invention, in which the means forming biocompatible culture support, located between the two membranes with different cutting threshold, is a bed of macrosupports.

FIG. 3 schematically represents the dynamic circulation of liquid media F1 and F3 for a gradient of pressure p3>p1 in an “ascending phase” within the culture chamber of a bioreactor of the invention, in which the means forming biocompatible culture support, located between the two membranes with different cutting threshold, is a bed of macrosupports.

FIG. 4 schematically represents a gradient of pressure p1>p3 in a “downward phase” within the culture chamber of a bioreactor according to the invention.

FIG. 5 schematically represents a gradient of pressure p3>p1 in an “phase ascending” within the culture chamber of a bioreactor according to the invention.

FIG. 6 represents a set-up schema of a bioreactor wherein the regulation-control unit is not represented.

FIG. 7 schematically represents the dynamic circulation of liquid media F1 and F3 for a gradient of pressure p1>p3 in a “downward phase” within the culture chamber of a bioreactor of the invention, in which the means forming biocompatible culture support, is a filtering membrane, so-called culture.

FIG. 8 schematically represents the dynamic circulation of liquid media F1 and F3 for a gradient of pressure p3>p1 in an “ascending phase” within the culture chamber of a bioreactor of the invention, in which the means forming biocompatible culture support is a filtering membrane, so-called culture.

### DETAILED DESCRIPTION OF THE INVENTION

European Patent No. 00115568.8 dated Jul. 19, 2000, from which this application claims priority under 35 U.S.C. § 119, is hereby incorporated herein by reference in its entirety.

The invention relates to a cell culture chamber with axis of synmmetry, containing at the same time the cells and the culture media comprising at least two filtering membranes with different cutting threshold and a means forming a biocompatible culture support located between two of the filtering membranes with different cutting threshold, said chamber being delimited by an envelope with axis of symmetry formed by an external lateral wall and two end walls.

The first membrane, so-called membrane of feeding has a cutting threshold chosen in the range of 0,01 μm to 7 μm which allows the biochemical exchanges within the culture chamber, by allowing the passage of the molecules of the nutriment media, such as proteins and macromolecules, while achieving a cell containment preventing the cultivated cells from leaving the zone of homeostasy and also preventing the passage of the contaminant particles, by being used, in particular, as a barrier to the bacteria, being able to contaminate said chamber.

Another filtering membrane, so-called dialysis membrane, has a cutting threshold of at most 15 KiloDalton (KDa) and allows the molecular containment of all the molecules having a molecular mass superior to 15 KDa. The presence of this filtering membrane within the culture chamber makes it possible to define with the first membrane a space of containment of the culture cells. Consequently, the filtering planar membrane with a cutting threshold of at most 15 KDa confines the cells in culture in the culture chamber, as well as the growth factors and the large proteins. According to one characteristic of the invention, at least one of the membranes has a cutting threshold preferably comprised between 0,2 μm and 4 μm, while the other has a cutting threshold preferably comprised between 10 and 12 KDa.

According to the invention, the number of membranes present in the culture chamber can be superior to two. In this case, the additional membranes have cutting threshold adapted to those of the two above mentioned membranes.

According to the invention, the filtering planar membranes with different thresholds of cut within the cell culture chamber are laid out perpendicularly to the axis of symmetry of said culture chamber.

According to the invention, the two filtering planar membranes with different cutting threshold can be mineral or organic membranes. For example, one can consider membranes made up of biocompatible organic polymers, cellulose or polysulphone.

Moreover, these two filtering membranes are distant from one another of at most about 25 mm and preferably of at most 20 mm, this distance being considered favorable for a good development of the cells as they are practically always in contact with the sources of nutrients and of oxygen.

The means forming a biocompatible culture support, allowing the adhesion of the cells in a state of culture, is confined between the two filtering membranes with different cutting threshold.

One of the means forming a biocompatible culture support according to the invention can be a bed of biocompatible macrosupports made of particles of various sizes which can be possibly agglomerated in a continuous block by sintering of the granular elements. This bed can have a thickness at most equal to the distance between the two filtering membranes with different cutting threshold. Said macrosupports bed has on the one hand a role of support of the cells in a state of culture and on the other hand a mechanical role of maintaining the required space of cell containment, arranged between the two filtering membranes with different cutting threshold.

According to the type of cells cultivated within the culture chamber of the invention, the type of biocompatible macrosupports will be chosen in an appropriate manner and will be of adequate size.

The macrosupports implemented between the two membranes of the culture chamber can have a cylindrical or spherical or still polyhedral shape such as for example machined massive blocks.

According to the invention, the macrosupports can be of mineral origin (such as, for example, coral) or of metallic origin (such as titanium and its alloys for example) or still made of biocompatible polymers.

On a purely illustrative basis, in the case of a culture of hematopoietic cells for an application for a bone-marrow graft on the one hand and in the case of a osteoblasts culture with an osseous rebuilding aim on the other hand, the macrosupports may be coral micro-beads. Such coral beads of desired granulometry appear to be suitable macrosupports for the applications mentioned above, due to their specific ability to be colonized by the hematopoietic progenitors. In the case of the osseous precursors, the coral beads can be furthermore metabolized, which would favor their use in rebuilding surgery.

Whereas, for example, in the case of a hepatic cells culture for an application of ex-vivo blood detoxification of an hepatic-insufficient patient by an extracorporeal biomass of hepatocytes, the most adapted macrosupports may be beads of polyamides, for example of nylon®, beads of fluoric polymers, for example of Teflon®.

Thus, the presence of macrosupports between said membranes and the fact that the filtering membrane with a cutting threshold of at most 15 KDa does not allow the passage of the cells in culture, as well as of the growth factors, allow the containment of the cells in culture, which adhere to said biocompatible macrosupports, within this space of cellular containment located between the two membranes with different cutting threshold.

Another means forming biocompatible culture support according to the invention can be a filtering membrane so-called culture M2, having particular characteristics distinguishing itself from the other membranes previously mentioned, that is to say the filtering membrane, so-called feeding membrane, with a cutting threshold in the range of 0.01 μm to 7 μm and the membrane, so-called dialysis membrane, with a cutting threshold of at most 15 KDa.

This membrane of culture, located between the two previously cited membranes, can lie on the membrane, so-called dialysis membrane, with a cutting threshold of at most 15 KDa.

Said membrane of culture can be of mineral or organic origin, which composition can vary according the different types of culture and the conditions of culture.

Thus, the membrane, so-called culture membrane, on which culture cells can multiply, can be modified by grafting of substrates or by co-culture of cells.

As non-restrictive illustrative examples, various types of modifications can be mentioned, which are:

the fixation of ligands for adherence molecules of glycoproteins types,

or the fixation of antibody,

or still the formation of a substratum from a first cellular type which constitutes the first culture of adherent cells, operated in an other bioreactor or in classic culture, then after the transfer of said in the culture chamber of said substratum, the setting up of a second cellular type, and optimization of the co-culture conditions. However, the formation of said substratum can also be done in a culture chamber according to the invention from a first cellular type, followed by a rinsing of said substratum, of the setting up of a second cellular type and the optimization of the co-cultures conditions,

or still the fixation of protein molecules.

The culture membrane which is also filtering, has a cutting threshold chosen in the range of 0.01 μm to 7 μm.

In the case of the implementation of such membrane, so-called culture membrane, as a means forming a biocompatible culture support according to the invention, located between the filtering membranes, so-called feeding membrane, with a cutting threshold chosen in the range of 0.01 μm to 7 μm and so-called dialysis membrane with a cutting threshold of at most 15 KDa, these two membranes can be supported by supports having appropriate meshes letting the passage for the dynamic liquid media F1, F2 and F3 intended to feed the cells culture chamber and to selectively extract the cultivated cells, the waste resulting from their culture and the nutrients in excess.

According to the need, said meshed supports intended to support the above cited membranes can be positioned in contact of one or the other sides, or both sides of said membranes, as well as in contact of one and/or the other sides of the membrane of culture.

The cell culture chamber as previously mentioned comprises an envelope with axis of symmetry formed by an external lateral wall and two end walls which can be assimilated to two flat bottoms located at each of the ends of said lateral wall.

This envelope with axis of symmetry can be made for example of a biocompatible polymeric material or of stainless steel. As biocompatible polymeric materials, one can quote for example polyolefins, polyamides, polyesters, or fluor polymers and others.

The feeding of the culture chamber in dynamic liquid media is carried out in a homogeneous manner because of the excellent distribution, by the internal sides of the end walls of the envelope, of the said media within said chamber thanks to inputs and outputs of these media judiciously positioned.

According to the invention, the internal sides of the two end walls constitute means of distribution of the dynamic liquid media.

According to a first type, the internal sides of the two end walls of the envelope with axis of symmetry are smooth, so that the distribution of the dynamic liquid media of feeding of the culture chamber in contact with the feeding and dialysis membranes can be realized in an homogeneous manner and naturally without stress.

According to a second type, which assures an organized feeding of the culture chamber in feeding liquid media, in contact with the feeding and dialysis membranes, the internal sides of the two end walls of the envelope with axis of symmetry are equipped grooves of distribution allowing the feeding of the culture chamber by two of the three dynamic liquid media. These grooves of distribution constitute a main network of grooves, so-called main network. This main network of grooves located on the internal side of each of the two end walls, on the opposite facing of the space of cell culture, can be divergent starting from the inlet tube arranged in said wall.

According to the invention, the main network of grooves, so-called main network, also called distribution network allows a homogeneous distribution of the two dynamic liquid media, which are forwarded to the culture chamber by means of biocompatible conduits which end(s) are connected at the level of the end walls. The number of grooves of distribution located on the side on the opposite facing of the space of cell culture of each of the two end walls is defined so as to obtain a good dispersion of the dynamic liquid media in the cell-culture chamber and will be determined depending on the shape of the envelope with axis of symmetry.

Said main network is completed by a secondary network, formed of grooves, so-called secondary grooves, which is shallower and of orthogonal approximately direction compared to the main network in order to favor the circulation between the zones of distribution delimited by the main network. The spacing out of this secondary network can vary so that the secondary grooves are brought closer and are more numerous on the opposite side of the input of the media flow, in order to facilitate the drainage and the evacuation of said media.

Two adjacent grooves of the main network constitute a zone of distribution and two adjacent grooves of the main network crossed by two adjacent grooves of the secondary network constitute a cell of distribution. The main network and secondary network form a fine “grid” network for the propagation of the dynamic liquid media. These two networks can for example form a meshed network in the form of a waffle.

The main network being located on the side on the opposite facing of the space of cell culture, has a certain height and a certain width that the person skilled in the art is completely able to define, knowing that the network of grooves must be in contact of the filtering planar membrane to ensure the cohesion of the whole system. Indeed, the volume arranged by the interdependent association of the grooves network and the membrane forms the grid of distribution of the liquid flow which can account for approximately 50% of the surface of the membrane.

Thus, for example, the main network can be constituted of main grooves having a depth of at most 5 mm and a width of at most 2 mm, the pitch comprised between two adjacent grooves of said main network can be of at most 2 mm. In the same manner, the secondary network can be constituted of secondary grooves having a depth of at most 2 mm and a width of at most 2 mm, the pitch comprised between two adjacent grooves of said secondary network gradually decreasing from the side of input of the media flow towards the output of said media flow. So, in its distal part—that is to say on the side of output of the media—the pitch of the secondary network can be of at most 2 mm.

Consequently, the end walls can be considered as doing a fine pattern of cells, for example of waffle type, onto which the membranes with different cutting threshold come to take support or are adhered by a biocompatible adhesive of type polymeric adhesive.

The envelope with axis of symmetry appears as formed of an external lateral wall and of two end walls.

The external lateral wall can be formed of at least three parts of same section, each having an adequate height, which can be identical. These at least three parts of wall constitute the external walls of at least three superimposed modules (C1, C2, C3), of which two (C1 and C3) receive the end walls of the envelope with axis of symmetry of the culture chamber, the third module (C2), disposed between the two latter, receiving at one of its ends the filtering planar membrane (M1), so-called feeding membrane, with a cutting threshold comprised between the range of 0.01 μm to 7 μm and at its other end the filtering planar membrane, so-called dialysis membrane, with a cutting threshold of at most 15 KDa.

The at least three modules are superimposed and are connected to each other in an impervious way due to impermeability joints, by means of appropriate fixing, such as for example, by adhesive bond, mechanical assembly by screw or other.

The form of the envelope with axis of symmetry of the cell-culture chamber, according to the invention, can be selected in a suitable manner in order to facilitate, for example, a stacking of several cell-culture chambers on a support. The person skilled in the art is once again capable to choose the form of the envelope with axis of symmetry of said culture chamber according to the use intended. For example, an envelope with axis of symmetry with section of circular or polygonal form can be considered.

It should be noted that a stacking of modules for chambers of culture can be carried out on the condition that the modules have the same form in order to obtain a stable stacking of these modules on an adequate supporting means.

For example, according to an embodiment of a culture chamber by stacking of several modules, a hexagonal form, for example, can facilitate the successive stacking of these modules on an appropriate base having for example six columns. In this case, the six columns of the base having the function of support for the stacking of these modules can also be used for example as feeding and disposal conduits of the dynamic liquid media.

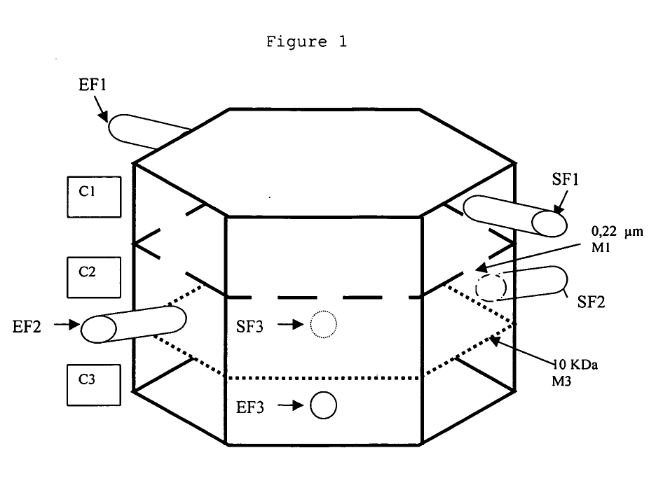

The feeding in dynamic liquid media of the culture chamber according to the invention is carried out by a system with three dynamic liquid media, namely a system with three flows of distinct media (FIGS. 1 to 8).

A first dynamic liquid media noted (F1), entering and outgoing by biocompatible tubes connected into the lateral wall of the module (C1) close to one of the end walls of the envelope with axis of symmetry (for example the wall of the upper end), feeds the culture media in nutrient elements (also called rich nutrient media), this media being rich in growth factors. This first dynamic liquid media is composed of elements necessary to the culture of the cells such as for example of proteins, oligo-elements, glucose, water and of growth factors, and feeds the culture media in fresh nutritional media.

A second dynamic liquid media noted (F2) entering and outgoing by biocompatible tubes connected into the lateral wall of the module (C2), forming a portion of the envelope with axis of symmetry of the culture chamber, can present three distinct functions according to the uses considered.

According to a first function, this second dynamic liquid media (F2), entering through a biocompatible tube connected to the level of the side wall of the culture chamber of the module (C2), is used to introduce into said chamber the cells intended to be cultivated and to recover the said cells cultivated within said module of the chamber after their culture (hematopoietic cells or hepatic cells for example).

According to a second function, this second dynamic liquid media can have the function of genes transfer. Indeed, at the time of the introduction of the culture cells in suspension in the module (C2) of the culture chamber by means of this second dynamic liquid media, viral particles contained in the said liquid media can fix themselves to the cells in suspension at the level of their membranes and thus to allow the desired-transfer of genes. This second flow of media can make it possible to obtain the desired culture cells, genetically modified, that one wishes to cultivate. Indeed, this media transports the vectors of gene transfer and allows their setting in contact with the cells so as to establish a membrane-fusion between the target cell and the vector of gene transfer. These vectors of gene transfer are of any nature. For the purpose of illustration, the viruses such as for example the adenoviruses, the retroviruses, the liposomes, of the plasmidic complexes can be cited.

Such a marking of the cells (by injection of vectors of gene transfer) realized out of the human body can make it possible to target with precision the tissue to genetically modify. Moreover, for example, a synthetic virus of single use to target the gene transfer on a cellular population of therapeutic interest can be used.

This virus, characterized in that the genes coding for the envelope and those which convey the genetic information are separated, is unable to reproduce itself inside the cells after having transferred the desired genetic information. Thus the risks of generation of a recombining virus starting from the used synthetic virus and from a wild virus which could be present in the patient, are limited.

According to a third function, this second dynamic liquid media (F2) can have the role of rinsing flow for the inhibiting macromolecules present within the space of cell containment. Indeed, the cells put into culture can have undergone a stress before their harvest or during their pre-inoculation processing or during their inoculation in the culture chamber. This stress can induce the production (by said cells) of proteins which will inhibit their capacity to multiply themselves by making them evolve in a state of dormancy, called quiescent state. During this state, said cells are not any more in physiological state to respond to the stimulation, carried out by the growth factors, by multiplying themselves.

In the case of the hematopoietic cells, one can refer to the so called “radio-induced” stress which is caused by an exposure to ionizing radiation (on purpose in the case of radio-therapy use or accidental) and results later on in a stress. This type of stress involves the production of inhibitors such as for example “Transforming Growth Factor-beta” or “Tumor Necrosis Factor-alpha”. These inhibiting molecules being cytokines as well as the growth factors brought for the culture of hematopoietic cells, are thus confined by the membranes with different cutting threshold of the culture chamber.

To carry out a suitable culture of the cells, it is necessary to eliminate from the culture media and so from the culture chamber these inhibiting molecules. The second dynamic liquid media in its third function is consequently used to rinse the zone of cell containment in order to eliminate said inhibiting molecules.

A third dynamic liquid media noted (F3), entering and outgoing by biocompatible tubes connected into the lateral wall of the module (C3) close to the second of the end walls of the envelope with axis of symmetry (for example the wall of the lower extremity) feeds the culture media in basic nutritive media (also called basic-regenerating nutritive media). This basic nutritive media is a nutrient media completely deprived of growth factors. Such a basic media is consequently composed of glucose, water, oligo-elements such as, for example, of the vitamins and minerals, of dyes in order to evaluate the pH of said media and of proteins of albumin type.

According to the invention, this cell culture chamber intended to be fed by the system with three distinct dynamic liquid media (F1, F2, F3) (still called triple flow) has for characteristic three inlet and outlet couples of said media. Two of these couples (F1, F3) are connected close to the end walls in order to feed the modules (C1) and (C3) and to allow their distribution in contact of the filtering planar membranes with different cutting threshold. The third couple (F2) is connected to the external side wall at a level being located between the two filtering planar membranes of different separation levels (cutting threshold), that is to say connected onto the module (C2).

The shifted location of these inlet and outlet couples of the media onto the envelope delimiting the culture chamber makes it possible to obtain an homogeneity of distribution of said liquid media within said chamber (see FIG. 1).

According to characteristics of the invention, the inlet and outlet tubes (or pipe) of the triple flow system are consequently distributed in an appropriate manner onto the envelope with axis of symmetry of the culture chamber.

In accordance with FIG. 1, the inlet biocompatible tube of the first flow noted EF1 is connected at a level close to the superior end wall of the envelope of the culture chamber, whereas the outlet tube noted SF1 of the first flow is also connected to the same end wall but at the opposite of the inlet tube.

The inlet biocompatible tube of the third flow noted EF3 is connected to a level of the inferior end wall of said envelope so that this tube is connected according to an angle of about 120° compared to the inlet tube the first nutrient flow (EF1) rich in growth factors in order to allow a good distribution of said media within the culture chamber.

In addition, the outlet biocompatible tube of this third flow noted SF3 is also connected to the same end wall but at the opposite of the inlet tube of the said flow (EF3).

In accordance with FIG. 1, the inlet biocompatible tube of the second dynamic flow noted EF2 is connected between the two filtering planar membranes noted (M1) and (M3), which respectively have a cutting threshold (separation level) of 0.22 μm and 10 KDa, at the level of the external lateral wall of the envelope according to an angle of about 60° compared to the inlet tube of the first flow (EF1). The outlet biocompatible tube noted SF2 is connected to the opposite of the inlet tube of the said flow on the side wall.

Consequently, the three couples of inlet and outlet of the dynamic liquid media are positioned in three vertical planes passing by the axis of symmetry of the envelope, these planes being shifted by an angle of about 60° between the first inlet and the second inlet, and of an angle of about 120° between the first inlet and the third inlet of the dynamic liquid media, the outlets of the said liquid media being in the same angular dispositions.

The present invention also concerns a bioreactor comprising the cell culture chamber previously described. This culture chamber, by means of these three inlet and outlet couples of the dynamic liquid media, is connected through connection conduits to the feeding tanks and/or to disposal tanks of said chamber.

The bioreactor according to the invention further comprises means of regulation of the conditions of culture and of control of the mass transfer of said dynamic liquid media, connected to the regulation-control unit of said bioreactor.

Thus, this bioreactor comprises in addition to the culture chamber “a unit of regulation also called unit of control” which allows the control and the regulation of the pH, of the oxygen concentration and the temperature of the culture media. The “regulation-control unit” can automatically process the complete operation of the bioreactor.

This regulation is of the numerical P.I.D type. (Proportional, Integral and Derived numerical regulation) and the data returned by the various sensors can be recorded and analyzed by computer data processing means.

So, the bioreactor of the invention containing the culture chamber previously mentioned, is organized in interchangeable functional unit modules dimensioned for a certain value of energy or mass transfer (such as, for example, aeration modules (ventilators), thermal exchangers), constituting a modular unit which can be adjusted by juxtaposition of identical functional units positioned in series and/or in parallel, and equipped with a programmable regulation-control unit.

The regulation-control unit receives the totality of the information related to the dynamic liquid media F1, F2, F3, by means of control and of regulation, as well as the information related to the various tanks, pumps, valves and pressures existing in the zones-modules C1, C2, C3 of the culture chamber, processes said information and dispatches the necessary functioning order signals.

So, it is possible to modify on request the internal characteristics of the culture chamber and to change the parameters and the programs of regulation contributing to the homeostasy of the media in order to adjust to the cultivated cellular type.

The regulation unit can also incorporate a crystalline cell of measurement of infra-red spectrum, which makes it possible to measure the concentration of certain aqueous solutions in the culture media by spectrum analysis. It is then possible to follow “on line” and “in real time” the concentrations of glucose and of lactate.

Thus, the temperature of the culture media can be fixed at a set point comprised between 30 and 38° C. During the cell multiplication, the pH of the culture media can be fixed at a set point comprised between 6.5 and 7.7. The regulation-control unit makes it also possible to regulate the air flow and the CO₂ rate which by its dissolution gives the amphoteric HCO₃⁻ which buffers the media and contributes to the stability of pH.

According to one of the characteristics of the bioreactor of the invention, the inlet and of outlet tubes (of the chamber) of first dynamic liquid media F1 are connected by connection conduits to a vessel R1 of nutrient media rich in growth factors according to a circuit operating in closed loop allowing the recycling of the growth factors necessitate for the development of the cell in culture. This vessel R1 can have the role of an expansion vessel.

According to one of the characteristics of this bioreactor, said connection conduit between the inlet tube of the first rich nutrient media F1 of the chamber and said vessel noted R1 of this media is equipped with a pump P1 making it possible to control and adjust the volume and the flow of the media F1 circulating in closed loop in the culture chamber.

The closed loop conveying the rich nutrient flow F1 is equipped with an air purge which is located on the expansion vessel R1, this air purge is fitted with a filter with a cutting threshold (separation level) of 0.22 μm guaranteeing the asepsis of the media. In the same way, this loop is equipped with an electromagnetic valve V1 controlled by the programmable regulation-control unit, allowing the pressure balance with the atmospheric pressure on request. The expansion vessel R1 is also provided with sensors of high level and of low levels of liquid media being used to set off the inversion of the circulation of the flows.

According to another characteristic of the bioreactor of the invention, the inlet tubes of the third basic nutritive media (EF3) of the culture chamber is connected by a connection conduit to a tank noted R3 of this media, while the outlet tube (SF3) is connected by a conduit to disposal (container for the recovery of waste).

According to the invention, the connection conduit connected to the inlet tube of third dynamic liquid media EF3 mentioned above is equipped with a pump P3 whereas the conduit connected to the outlet tube of said flow is equipped with a valve V3 possibly controlled by the regulation-control unit, this unit making it possible to regulate and control the volume and the flow of said third media F3 circulating in open circuit in the culture chamber.

According to the invention, an aerator device of the rich nutrient media F1 and an aerator device of the basic nutritive media F3 can be envisaged on one and the other of the two circuit in closed and open loops, place respectively between the inputs of said dynamic liquid media F1 and F3 in the cell-culture chamber and the vessels/tanks R1 and R3.

According to the invention, a thermal exchanger of the rich nutrient media F1 and a thermal exchanger of the basic nutritive media F3 can be envisaged on each of two circuits at the entrance of said dynamic liquid media F1 and F3 before accessing the cell-culture chamber.

These devices make it possible to maintain the homeostasy of the culture chamber by pre-conditioning the flows of media which penetrate into it in order not to enter into the culture chamber an element of volume of culture media which could induce a brutal disturbance of its physicochemical parameters.

According to another characteristic of the bioreactor of the invention, the inlet tube of second dynamic liquid media EF2 of culture chamber, located at the level of side wall of the envelope, is connected by a connection conduit to a tank noted R2 containing the second dynamic liquid media selected in accordance with the function which will be attributed to said media, that is either to feed the culture chamber in cells intended to be cultivated and to discharge said chamber after culture, or to carry out a gene transfer, or to have a role of rinsing flow intended to discharge the culture chamber of molecules inhibiting the cells to cultivate.

According to the attributed function to the second dynamic liquid media of the culture chamber, the outlet tube SF2, located at the opposite of the inlet tube EF2 on said wall, will be connected:

by a connection conduit to a recovery tank which will recover the cells after culture (so called harvesting container) or to a waste disposal (container for the disposal of the inhibiting molecules) according to a circuit in open loop, or

by a connection conduit to a tank containing the media provided with the suitable vectors of gene transfer according to a circuit in closed loop.

According to an other characteristic of the bioreactor of the invention, one or more media-exchange functional modules, also called dialysis or ultra-filtration units, can be added to purify the nutrient media F1 at the outlet of the culture chamber. This or these media-exchange functional modules can be located at the exterior of the culture chamber, and can be assembled in series on the connection conduit of outlet of the circuit in closed loop of the first nutrient rich in growth factors media F1 and are traversed in counter current by the basic nutritive media F3 in open loop.

In the case of the use of two functional modules connected in series in an ad hoc way on the closed loop F1, one can use two different thresholds of cut (separation levels), for example, of 10 KDa and 30 KDa.

One may, while injecting the flow resulting from module 10 KDa into the module of 30 KDa, and while recovering the permeate—that is to say the fraction of liquid which has crossed the membrane with cutting threshold 30 Kda—to re-inject said permeate into the loop F1, to carry out a sorting of protein present in the media, between 10 and 30 KDa which correspond with the size of the growth factors.

Such additional functional modules can be useful when, for example, the growth factors are produced by supporting cells in a first additional culture chamber, and that one wishes to pre-condition this media (i.e. to recover the produced growth factors, without recovering the cellular wastes generated by these cells of support) to be able to re-use it in the loop F1 in order to stimulate the cells, said of therapeutic interest, present in the main culture chamber.

It should be noted that the process used to cultivate hematopoietic and/or blood cells, can use cells of support, so-called stromales cells. These stromales cells can be genetically modified in order to produce human cytokines with levels of expression such as they can provide, to some extent, with the requirements in cytokines of the culture.

In addition, it should be noted that the envelope with axis of symmetry making it possible to obtain a homogeneity of distribution of the nutriments with an optimal dispersion has the advantage to be sterile as well as all the elements of the bioreactor in contact with the cells and the culture media.

Indeed, the sterilization of the bioreactor is carried out by pressure-sealing at 121° C. during 20 minutes for the entire apparatus, including the bottles of tanks. The connection conduits, the vessels, tanks and other tight proof elements are made out of biocompatible materials being able to support without damage ten cycles of sterilization in the case of use in laboratory. On the other hand, the whole unit comprising the culture chamber, connection conduits, vessels and tanks containing the media and others will constitute a kit of culture of single use when used in human related clinical conditions.

In addition, the reconditioning of the bioreactor after a cellular culture, within the scope of a use of laboratory type, is made by a proteic digestion with a molar hydrochloric acid solution followed by an ultra pure rinsing and by a re-sterilization.

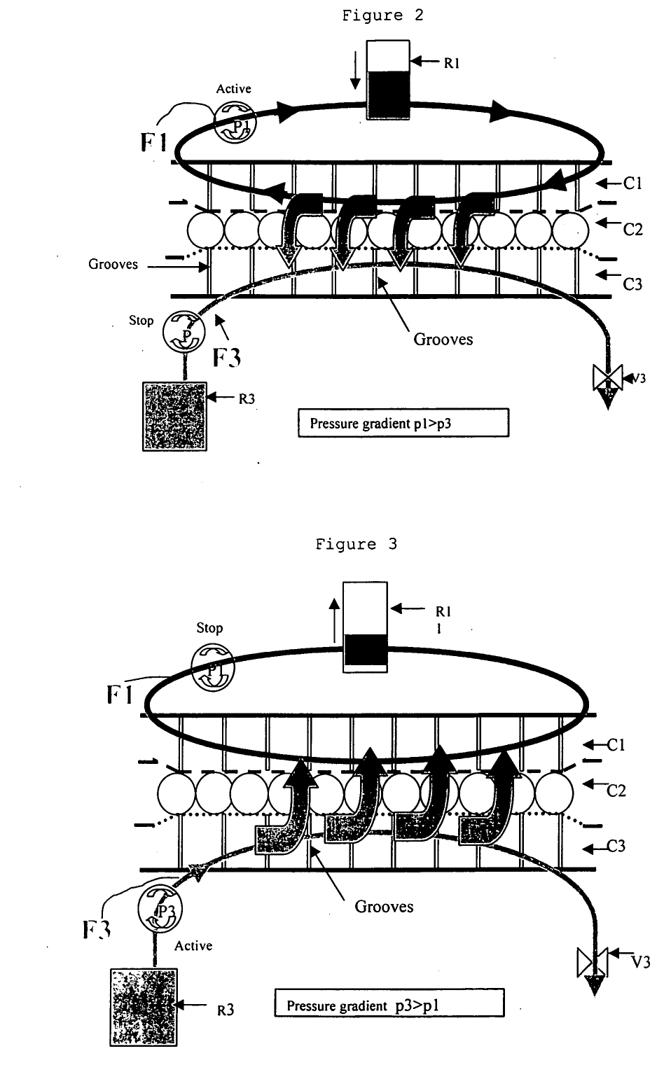

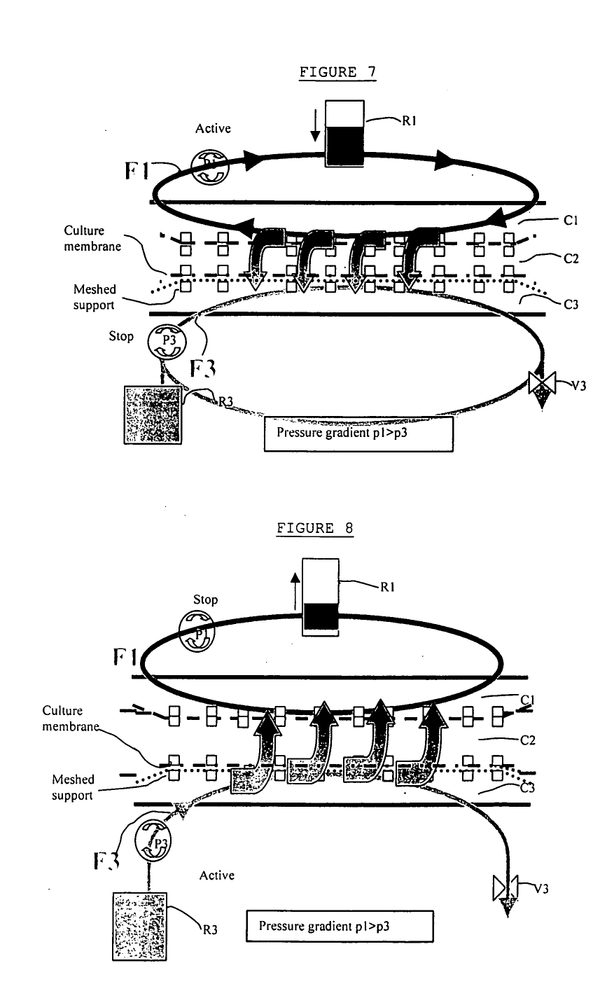

According to the operating mode of the bioreactor of the invention and in accordance with the FIGS. 2 and 7, the rich nutrient media F1 is introduced at the level of the superior end wall of the envelope in the zone (C1) (module or compartment C1) of the culture chamber comprised between said wall and the filtering planar membrane with cutting threshold (separation level) of the order of 0.01 μm to 7 μm, by the opening of the pump P1 located on the connection conduit which connects the culture chamber with the vessel R1 containing said media F1 whereas the pump P3 located on the connection conduit which connects the culture chamber with the tank R3 containing the basic nutritive media F3 is stopped.

Furthermore, the pressure p3 prevailing in the zone noted C3 (module or compartment C3) of the culture chamber located between the inferior end wall of the envelope and the filtering planar membrane with cutting threshold of at most 15 KDa, is regulated by the valve of back-pressure noted V3 located on the conduit which connects the culture chamber with the disposal so that the pressure p1 prevailing in the zone C1 of the culture chamber is superior to the pressure p3 and that the pressure p2 which prevails in the zone noted C2 (module or compartment C2) is comprised between p1 and p3 according to a gradient of pressure.

So, during the introduction of first dynamic liquid media F1 in the culture chamber achieved by the opening of the pump P1, the growth factors of the nutrient media pass into the zone noted C1 and through said membrane with cutting threshold of the order of 0.01 μm to 7 μm of culture chamber, but are retained by the filtering planar membrane with cutting threshold of at most 15 KDa, which functions as a barrier for the passage of the growth factors and the large proteins.

Consequently, the growth factors can migrate on each side of the filtering planar membrane with cutting threshold (separation levels) of the order of 0.01 μm to 7 μm ensuring their role of stimulation of the growth and/or control of the differentiation for the cells in state of culture confined between the two planar membranes of the culture chamber defining the zone C2 (module or compartment C2) of said chamber.

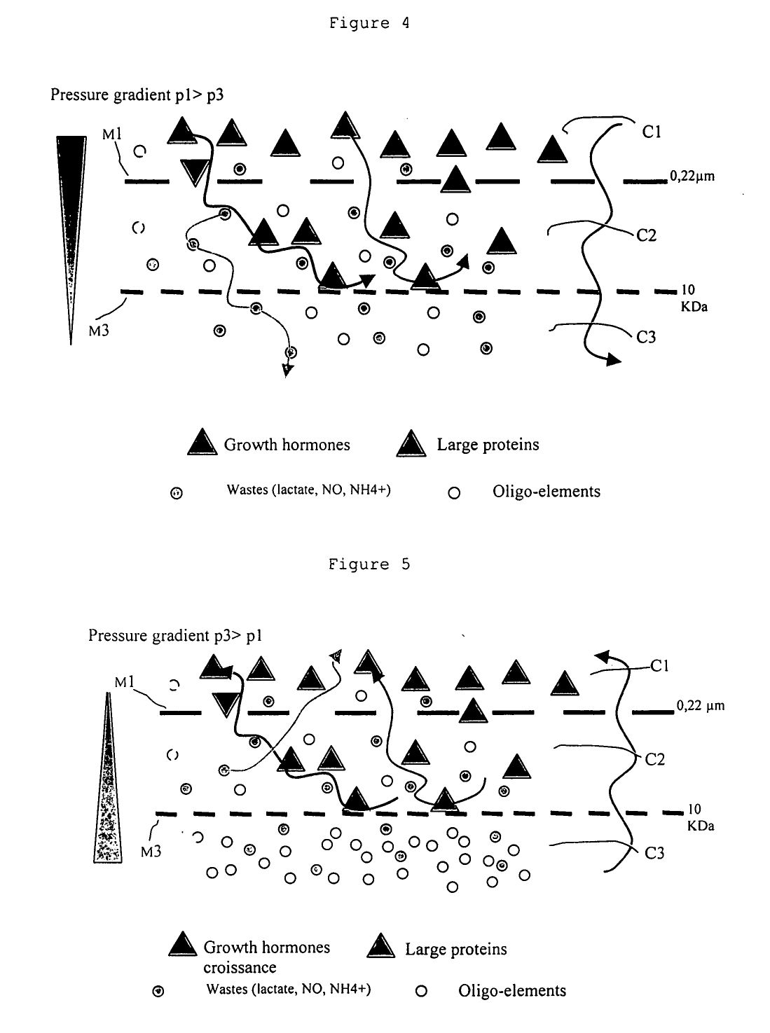

In accordance with the FIG. 4, the filtering planar membrane M3 with cutting threshold (separation level) of at most 15 KDa confines the growth factors and the large proteins of the fresh nutritive media F1 in the zones C1 and C2 of the culture chamber. On the other hand, the oligo-elements of said nutritive media F1 and the wastes of small sizes (such as, for example, NO, NH₄⁺, lactate and others) generated by the culture of the cells confined in the zone C2 of said chamber, are drained towards the zone C3 where they are carried towards the disposal outlet. The total trans-membrane flow is consequently directed from the zone C1 towards the zone C3 of the culture chamber (see FIGS. 2 and 7).

In this operating mode of the bioreactor according to the invention, in a so-called “downward phase”, the fresh nutritive media F1 circulating in closed loop loses volume because of the drainage of the aqueous solution towards the zone C3 and in particular towards the disposal outlet. Consequently, the volume of nutrient media F1 in the vessel R1 decreases. The growth factors and the large proteins of the fresh nutritive media F1 being confined in the zones C1 and C2, the wastes are purged from the zones of the culture chamber towards the zone C3 and are drained towards the disposal outlet by means of the open valve V3.

As soon as the volume of the vessel or expansion vessel R1 of the closed loop conveying the fresh nutritive media F1 reaches a certain low level, the regulation-control unit of the bioreactor programmed according to a particular sequence reverses the circulation of flow. So the pump P1 which worked is stopped while the pump P3 which was idle is started. And, the open valve V3 is then closed.

Consequently, the pressure p1 prevailing in the zone C1 of the cell-culture chamber becomes inferior to the pressure p3, whereas the pressure p2 prevailing in the zone C2 is comprised between p1 and p3, according to a gradient of pressure reversed compared to the preceding operating mode. The total trans-membrane flow is then directed from the zone C3 towards the zone C2 of the cell-culture chamber (see FIGS. 3 and 8).

In this operating mode of the bioreactor and in accordance with FIGS. 3, 5 and 8, mode called “ascending phase”, the inversion of flow within the cell-culture chamber allows the fresh nutritive media F1 circulating in the cell-culture chamber to reload itself in fresh nutritive elements coming from the third dynamic liquid media F3 and to compensate for the losses, amongst other things water, caused by the “downward phase”. Indeed, this basic (regenerating) media F3 re-feeds the cellular culture chamber in glucose, water, oligo-elements.

As soon as the volume of the vessel or expansion vessel R1 of the circuit in closed loop conveying the fresh nutritive media F1 reaches a certain high level, the system of control of the bioreactor programmed according to a particular sequence reverses again the circulation of flow.

Thus, the growth factors because of circulation in closed loop of the rich nutrient media F1, that is to say of the first dynamic liquid media of which they form parts, are confined in the closed loop and the compartments C1 and C2, and their concentration oscillates between a concentration C₀ and C₀+/− C. The rich culture media is thus recycled and the use of the growth factors optimized thanks to the culture chamber and the bioreactor of the invention.

This flow inversion system within the culture chamber makes it possible to create trans-membrane flows subjected to low hydrodynamic stresses compatible with the fragility of the cultivated cells. Thus, a system of “laminar” slow flows is obtained. This inversion of flows can also prevent the plugging of the filtering membranes whose trans-membrane pressure loss (index measuring the permeability of the membrane) could be controlled in real time by electronic pressure gauges placed on each flow of dynamic liquid media.

In the operating mode of the bioreactor of the invention, the second dynamic liquid media (F2) entering and outgoing on the level of the external side wall, between the two filtering planar membranes of the culture chamber, circulates within said chamber according to a circuit in open loop or in closed loop according to the function allocated to the flow, the pumps P1 and P3 being stopped, and its inlet and outlet configuration is fixed according to the function one has set.

According to the operating mode of the bioreactor, when the dynamic second liquid media F2 in its first function is used to inoculate the cells to cultivate in the culture chamber and to discharge them after culture, it operates in an open loop just as it does when it is used in its third function to rinse the chamber in order to eliminate the inhibiting molecules. On the other hand, when the dynamic liquid media F2 has a role of genes transfer, it operates in closed loop during the phase of inoculation and of incubation.

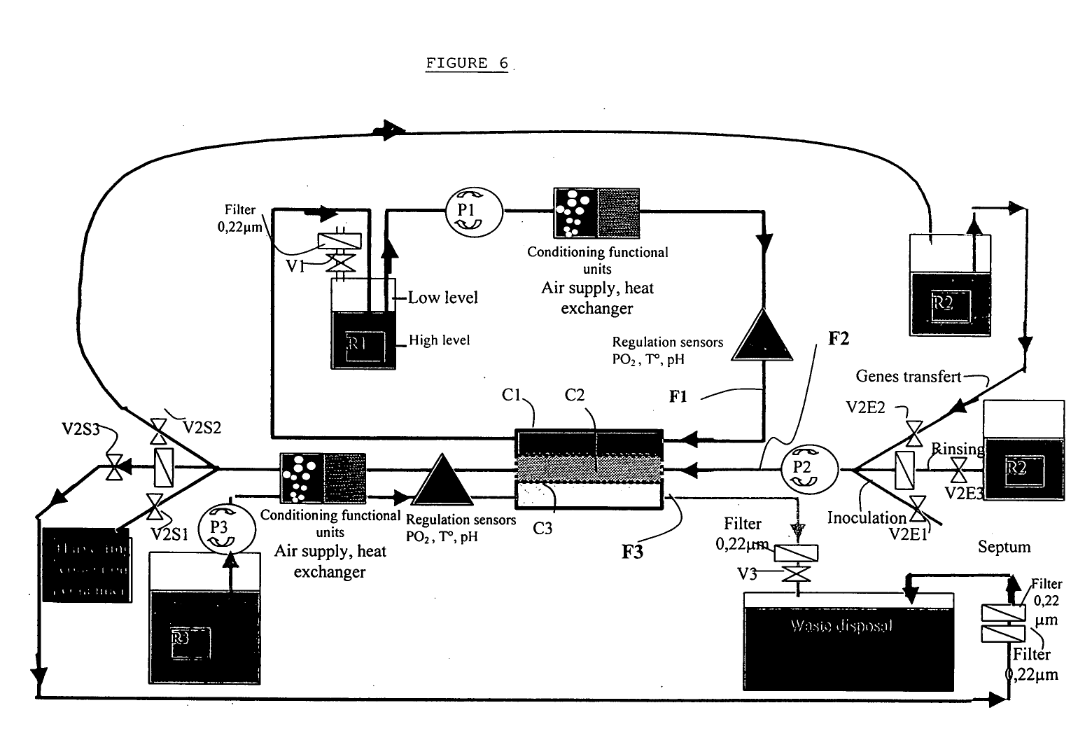

In its first function and according to the operating mode of the bioreactor in accordance with FIG. 6, the second dynamic liquid media F2 containing the cells to cultivate is inoculated in the zone C2 of the culture chamber by a syringe placed through a biocompatible septum positioned on one of the three segments of the inlet tube EF2 (of the external wall side) of which the electro-valve V2E1 is open, the electromagnetic valves V2E2 and V2E3 being closed. While the inoculation of the said media takes place, the three electromagnetic valves V2S1, V2S2 and V2S3, located on the three segments of the outlet tube SF2 located at the opposite of the inlet tube (pipe) EF2, are closed.

During the recovery of the cells after culture, the two electromagnetic valves V2E2 and V2E3 of the inlet tube EF2 are closed, the electro-valve V2S1 located on one of the three segments of the outlet tube SF2 connected to a connection conduit to the tank of recovery of the cultivated cells, is open whereas the electromagnetic valves V2S2 and V2S3 of the two other segments of the outlet tube SF2 are closed.

The regenerating basic media is then pumped by the starting up of the pump P2 from the tank R2 and is used to purge the contents of the compartment C2 (said content being situated between the two membranes with different separation levels) in the cellular collection container. An enzymatic treatment of trypsin and/or collagenase and/or DNAse type can be employed in order to de-structure the extra-cellular matrix produced by the cells during their culture in order to facilitate their anchoring. Within the framework of a culture of hematopoietic cells on a macrosupport of the coral type, a slight acidification of the media with pH=6.0 or pH=6.5 will make it possible to dissolve the coral and to recover the hematopoietic cells after rinsing.

In its third function and according to the operating mode of the bioreactor, when the second dynamic liquid media F2, containing the elements necessary to the rinsing of the cellular containment space, functions in an open loop according to the same principle of closing and opening of the electromagnetic valves described above with the exception that the inlet tube is not equipped with a biocompatible septum but is connected by a conduit of connection to the tank R2 containing said selected media F2.

When introducing the rinsing media F2 in the zone C2 of the culture chamber at the level of one of the three segments of inlet tube EF2 (of the external side wall) of which electro-valve V2E3 is open, the electromagnetic valves V2E1 and V2E2 are closed.

When introducing and recovering of the said rinsing media, the two electro-valves V2S1, V2S2 located on the two segments of the outlet tube SF2 are closed, the electro-valve V2S3, located on the other segment connecting the outlet tube SF2 being open, the rinsing flow continuously circulating in an open loop. The output of electro-valve V2S3 can be connected on a tube connected to a sewer disposal. The sewer disposal is constituted of a tank of sterilized media, the sterile environment being equally required for whole of the tubes (pipes) of the bioreactor, of its tanks, its functional modules and of the culture chamber, which thus guarantees the sterility of the flow. Two filters of 0.22 μm can be added to this sewage disposal in order to increase the security and to guarantee the sterility, namely when it is necessary to change “at hot-setting” the sewer because of an overflow.

In its secondary function and according to the operating mode of the bioreactor in accordance with the FIG. 6, the second dynamic liquid media F2 containing the vectors of gene transfer is introduced into the zone C2 of the culture chamber by the opening of the electro-valve V2E2 located on one of the three segments of inlet tube EF2, the electro-valves V2E1 and V2E3 being closed.

During the introduction of said media, the two electro-valves V2S 1, V2S3 located on two segments of the outlet tube SF2 are closed. This media F2 containing the vectors of gene transfer circulates in closed loop during the time necessary for the inoculation and incubation phases. When the procedure of transfer of genes is finished, the loop F2, switches again to its rinsing mode such as described above, in order to rinse the possible residual vectors. Then, the rinsing being finished, the culture continues according to the alternation of the “ascending” and “downward” phases of the flows F1 and F3.

It should be noted that one of the non desired side effects happening during the inversion of the flow of the downward phase towards the ascending phase is the possibility that the media F1 is slightly contaminated by the wastes coming from the zones C2 and C3. But as it appears the phenomenon of dilution makes this possible contamination negligible. Moreover, the proximal part of the conduit of connection connecting the reservoir of the basic media F3 to the culture chamber is loaded with fresh media F3, that is to say coming from said tank, only the distal part of the conduit connecting the culture chamber to the sewage disposal, that is to say close to the valve V3, is loaded with wastes.

The culture chamber and the bioreactor according to the invention can be used for the in vitro culture of animal cells and, for this reason, the invention relates to the medical field. On an illustrative basis, the culture chamber and the bioreactor according to the invention can be used for the production of hematopoietic cells or hepatic cells.

The present invention thus finds its application for extracorporeal culture of cells of the osseous marrow.

The hematopoiesis is the physiological processing which makes it possible to renew all the figured elements of blood (mature blood cells having a limited life-time) by the multiplication and differentiation of a multi-potent original primitive cell (called original cell) able to engender all the cellular types, blood cells still called figured elements of blood. This process is under the control of hematopoietic growth factors, therefore called cytokines.

The grafting of immature hematopoietic cells, called hematopoietic pro-genitors, onto a patient subjected to an accidental aplasia or aplasia resulting from an anti-cancer treatment is a means of being able to restart the activity of the injured osseous marrow in order to initiate the restart of the process of hematopoiesis.

This processing implies that pro-genitors—cells at an early stage of development—are taken from the patient by cytapheresis or directly by puncture of the osseous marrow, and are re-injected after culture so that these cells can reconstitute the blood and immunizing system of the patient (in the case of a complete aplasia) or compensate for the morbid period of transitory pancytopenia (phase of neutropenia, lymphopenia and thrombopenia) in order to allow the endogenous hematological resumption of the aplasied victim (in the case of accidental irradiation).

In the same way in the case of an acute hepatic insufficiency involving a vital risk in a very short term, it is necessary to compensate for the defective hepatic functions of the patient by a processing of extracorporeal blood detoxification requiring so a great number of hepatocytes to carry out the necessary metabolic functions. The culture chamber as well as the bioreactor described above can allow the production of hepatic cells in a sufficient number to obtain an effective treatment and a quick detoxification compatible with the hemo-dynamic constraints which poses the extracorporeal circulation of the blood of the patient.

On an illustrative basis, a useful bioreactor of a volume which can vary from 50 to 100 ml allows to carry out an extracorporeal culture of animal cells over a period of about ten days within the framework of the production of a cellular biomass with the intended use of grafting and/or as palliative means in the case of a deficiency.

Within the framework of the reconstitution of a tissue model organized and arranged hierarchically, the duration of culture authorized by the bioreactor can reach several months.

For a grafting of cells of the osseous marrow type (hematopoietic cells), it is necessary that 10⁶ to 10⁷ of CFU (type of immature cells)/Kg of the weight of the patient result from the culture. This clinical threshold limit which conditions to some extent the success of the grafting, makes it possible to estimate to 10¹⁰ the number of cells resulting from the hematopoietic culture necessary for the grafting. The culture chamber(s) as well as the bio-reactor such as described can be dimensioned for this type of application.

The conditions of pH, temperature and partial oxygen pressure for a culture of the mentioned above cells will be of the order of 7.4 for a pH of growth, of the order of 37.5° C. for the temperature and a partial oxygen pressure (in % of saturation) equal to 15%.

The nutrient media is a synthetic media consisting of ultra purified or synthesized proteins and of oligo-elements (such as iron, selenium, transferrine, vitamins and others) and of a vital dye. This nutrient media is enriched by growth factors which are cytokines which concentration can vary from 10 to 100 ng/ml according to needs.

Moreover, the cytokines can be stabilized in an active biologically state in the presence of heparanes. The basic regenerating nutritive media is marketed under the following trade names, it can be MEM-alpha or RPMI1640 or still IMDM.

1. Culture chamber for extracorporeal culture of animal cells, delimited by an envelope with axis of symmetry, which is formed of an external lateral wall, of two end walls, and of inlets and outlets of the dynamic liquid media, characterized in that it comprises:
a) at least two filtering planar membranes M1 and M3 with different cutting threshold, perpendicular to the axis of symmetry;
b) between the membranes (M1 and M3) a means forming a biocompatible culture support allowing the adhesion of cells in state of culture;
c) two end walls constituting means of distribution of the dynamic liquid media;
d) three inlet and outlet couples of the dynamic liquid media (F1, F2, F3), intended to feed the cells culture chamber and to selectively extract the cultivated cells, the wastes resulting from their culture and the nutrients in excess, two of the couples being, for each of them, connected between one of the end walls and one of the membranes, the third couple being connected between the two filtering planar membranes.

2. Culture chamber for extracorporeal culture of animal cells according to claim 1, characterized in that one of the filtering planar membrane has a cutting threshold comprised between 0.01 μm and 7 μm, and the other filtering planar membrane has a cutting threshold of at most 15 KiloDaltons (KDa).

3. Culture chamber according to one or the other of claims 1 and 2, characterized in that one of the filtering planar membrane has a cutting threshold preferably comprised between 0.2 μm and 4 μm and the other filtering planar membrane has a cutting threshold preferably comprised between 10 and 12 KDa.

4. Culture chamber according to one at least of claims 1 to 3, characterized in that the two membranes with the different cutting threshold are distant from one another of at most 25 mm and preferably between 0.2 and 20 mm.

5. Culture chamber according to one at least of claims 1 to 4, characterized in that the means forming a biocompatible culture support allowing the adhesion of cells in state of culture is a bed of biocompatible macrosupports.

6. Culture chamber according to claim 5, characterized in that the bed of macrosupports has a thickness at most equal to the distance between the two filtering membranes with the different cutting threshold.

7. Culture chamber according to one at least of claims 5 and 6, characterized in that the macrosupports present between the membranes have a cylindrical, or a polyhedral or a spherical.

8. Culture chamber according to one at least of claims 5 to 7, characterized in that the constitutive material of the macrosupports is chosen among the group of mineral materials, metallic materials and polymers materials.

9. Culture chamber according to claim 8, characterized in that the constitutive material of the macrosupports is preferably chosen among the group constituted by coral, titanium and its alloys, polyamides, fluor polymers.

10. Culture chamber according to one at least of claims 1 to 4, characterized in that the means forming a biocompatible culture support allowing the adhesion of cells in state of culture is a filtering membrane of culture M2 located between the two filtering planar membranes M1 and M3 with different cutting threshold.

11. Culture chamber according to claim 10, characterized in that the filtering membrane of culture M2 lies on the membrane with cutting threshold of at most 15 KDa.

12. Culture chamber according to one of claims 10 and 11, characterized in that the filtering membrane of culture M2 is modified by grafting or co-culture of cells.

13. Culture chamber according to claim 12, characterized in that the modifying grafting of the filtering membrane of culture M2 concerns the fixation of ligands for adherence molecules of glycoproteins types, of antibody, of protein molecules.

14. Culture chamber according to claim 12, characterized in that the modifying by co-culture of cells is realized by formation of a substratum from a first cellular type which constitutes the first culture of adherent cells, followed by a rinsing of said substratum and the setting up of a second cellular type, and the optimization of the co-cultures conditions.

15. Culture chamber according to one at least of claims 10 to 14, characterized in that the filtering membrane of culture M2 forming a biocompatible culture support has a cutting threshold chosen in the range of 0.01 μm to 7 μm.

16. Culture chamber according to one at least of claims 1 to 4 and 10 to 15, characterized in that the filtering membranes M1 and M3 with different cutting threshold and the filtering membrane of culture M2 are supported by meshed supports.

17. Culture chamber according to one at least of claims 1 to 16, characterized in that the internal sides of the end walls of the envelope with axis of symmetry constitute means of distribution of the dynamic liquid media.

18. Culture chamber according to claim 17, characterized in that the internal sides of the end walls are smooth.

19. Culture chamber according to claim 17, characterized in that the internal sides of the two end walls are equipped of distributing grooves of the two dynamic liquid media F1 and F3.

20. Culture chamber according to claim 19, characterized in that the distributing grooves of the end walls are organized in a main network, these grooves being preferably divergent in the direction of the circulation of the dynamic liquid media.

21. Culture chamber according to one at least of claims 19 to 20, characterized in that the distributing grooves of the end walls are completed by a secondary network formed of secondary grooves which are slightly perpendicular to the grooves of the main network.

22. Culture chamber according to one or the other of claims 19 to 21, characterized in that the main and secondary networks constitute a grid network for the distribution of the dynamic liquid media.

23. Culture chamber according to one at least of claims 19 to 22, characterized in that the grooves of the main network and/or of the secondary network of the end walls are in contact with the membranes with different cutting threshold.

24. Culture chamber according to one at least of claims 19 to 22, characterized in that the grooves of the main network have a depth of at most 5 mm, and a width of at most 2 mm, and the pitch comprised between two adjacent grooves is of at most 2 mm.

25. Culture chamber according to one at least of claims 19 to 23, characterized in that the grooves of the secondary network have a depth of at most 2 mm and a width of at most 2 mm.

26. Culture chamber according to one at least of claims 1 to 25, characterized in that one of the two inlet and outlet couples F1 of the dynamic liquid media is connected between an end wall and the membrane M1 with a cutting threshold comprised between 0.01 μm to 7 μm and feeds the cells culture media with a nutrient media comprising growth factors, by passing through the filtering planar membrane with a cutting threshold comprised between 0.01 μm to 7 μm.

27. Culture chamber according to claim 26, characterized in that the inlet and outlet couple of the dynamic liquid media F1 is operating in closed loop.

28. Culture chamber according to one at least of claims 1 to 27, characterized in that the inlet and outlet couple of the dynamic liquid media F2 connected between the two filtering membranes serves the functions of introducing the cells to cultivate and of recovering said cultivated cells, of genes transfer and of recovery of the genetically modified cells, and of introducing a liquid of rinsing and of removal of the molecules inhibiting the cells growth in culture.

29. Culture chamber according to one at least of claims 1 to 28, characterized in that the other of the two inlet and outlet couples of the dynamic liquid media F3 is connected in an open loop at the level of the other end wall and feeds the culture media of the cells with a nutrient media free of growth factors, by passing through the filtering planar membrane with a cutting threshold of at most 15 KDa.

30. Culture chamber according to one at least of claims 1 to 29, characterized in that the three inlet and outlet couples of the dynamic liquid media are positioned in three vertical plans passing through the symmetrical axis of the envelope, these plans being shifted of an angle of approximately 60° between the first inlet and the second inlet, and of an angle of approximately 120° between the first inlet and the third inlet of the dynamic liquid media, the outlets of said liquid media being in the same angular positions.

31. Bioreactor for extracorporeal culture of animal cells comprising a culture chamber, delimited by an envelope with axis of symmetry, formed of an external lateral wall, of two ends walls and of inlet and outlet of the dynamic liquid media, and comprising means for achieving the circulation of said media in said chamber, characterized in that it comprises:
a) a culture chamber of said cells comprising at least two planar filtering membranes M1 and M3 with different cutting threshold, perpendiculars to the axis of symmetry, and that between said membranes with different cutting threshold a means forming a biocompatible culture support is located, allowing the adhesion of cells in culture, said chamber being delimited by an envelope with axis of symmetry comprising two end walls constituting means of distribution of the dynamic liquid media and three inlets and outlets couples of the dynamic liquid media F1, F2, F3, intended to feed the cells culture chamber and to selectively extract the cultivated cells, the wastes resulting from their culture and the nutrients in excess, two of the couples being, for each of them, connected between one of the end walls and one of the membranes, the third one being connected between the two planar filtering membranes;
b) circulation means of the first dynamic liquid media F1 operating in a closed loop and an expansion vessel R1 containing said media, said means being connected to said culture chamber;
c) circulation means of the second dynamic liquid media F2 operating in a closed loop or in an open loop depending upon the intended function dedicated to said media, and a tank R2 containing said media, these means being connected to said culture chamber;
d) circulation means of the third dynamic liquid media F3 operating in an open loop and a tank R3 containing said media, these means being connected to said culture chamber;
e) control, regulation and conditioning means for the dynamic liquid media linked to a regulation-control unit.

32. Bioreactor according to claim 31, characterized in that the circulation means of said liquid media F1, F2 and F3 are respectively equipped with pumps P1, P2 and P3 connected to the regulation-control unit.

33. Bioreactor according to claims 31 and 32, characterized in that the circulation means of the third dynamic liquid media F3 are equipped with a valve V3 connected to the regulation-control unit.

34. Bioreactor according to claims 31, characterized in that the expansion vessel R1 containing the dynamic liquid media F1 rich in growth factors is equipped with a filter having a cutting threshold of 0.22 μm and an electro-valve VI controlled by the regulation-control unit.

35. Bioreactor according to one at least of claims 31 to 34, characterized in that the expansion vessel R1 is equipped with sensors of high level and low level of liquid media which serve to activate the inversion of the circulation of the liquids within said culture chamber.

36. Bioreactor according to claim 31, characterized in that the conditioning means of dynamic liquid media comprise means of regulation in oxygen, temperature and in pH of said media.

37. Bioreactor according to claim 36, characterized in that the conditioning means of said media are functional unit modules, dimensioned for a certain value of mass transfer or thermal energy.

38. Bioreactor according to claim 37, characterized in that the functional modules consist of aeration modules, thermal exchangers, pH regulators, or still dialysis or ultra-filtration modules.

39. Bioreactor according to one at least of claims 31 to 38, characterized in that it comprises a culture chamber according to one at least of claims 1 to 30.

40. Bioreactor according to one at least of claims 31 to 39, characterized in that a pressure p1 prevails in the zone C1 of the culture chamber, located between the superior end wall and the filtering planar membrane with cutting threshold comprised between 0.01 μm and 7 μm of said chamber.

41. Bioreactor according to one at least of claims 31 to 40, characterized in that a pressure p2 prevails in the zone C2 of the culture chamber comprised between the two filtering planar membranes with different cutting threshold of said chamber.

42. Bioreactor according to one at least of claims 31 to 41, characterized in that a pressure p3 prevails in the zone C3 of the culture chamber located between the filtering planar membrane with cutting threshold of at most 15 KDa and the inferior end wall of the envelope of said chamber.

43. Bioreactor according to one at least of claims 31 to 42, characterized in that nutrient media rich in growth factors F1 is drained from the zone C1 of the culture chamber toward the zone C3 of said chamber when the pressure p1 is superior to the pressure p3, and when the pressure p2 is comprised between the pressure p1 and p3 in the culture chamber.

44. Bioreactor according to one at least of claims 31 to 42, characterized in that nutritive base media free of growth factors F3 is drained from the zone C3 of the culture chamber toward the zone C1 of said culture chamber when the pressure p3 is superior to the pressure p1 and that the pressure p2 is comprised between the pressure p1 and p3 in the cell culture chamber.

45. Bioreactor according to one at least of claims 31 to 43, characterized in that the opening of the pump P1 allows the feeding of the culture chamber in nutrient media rich in growth factors F1, the pump P3 being stopped, and that the valve V3 regulating the back-pressure is in such a way that the pressure p1 prevailing in the zone C1 of said culture chamber is superior to the pressure p3 prevailing in the zone C3 of said chamber, knowing that the pressure p2 prevailing in zone C2 is comprised between the pressures p1 and p3.

46. Bioreactor according to claim 45, characterized in that the regulation-control unit programmed according to a specific sequence inverts the direction of the circulation of the flow by stopping the pump P1 and starting the pump P3 as soon as the volume contained in the expansion vessel R1 reaches a low level.

47. Bioreactor according to claim 45, characterized in that the valve V3 is closed.

48. Bioreactor according to claims 46 and 47, characterized in that the nutritive base media free of growth factors F3 allows the re-feeding of the culture chamber wherein the pressure p3 in the zone C3 of said chamber is superior to the pressure p1 prevailing in zone C1, knowing that the pressure p2 prevailing in zone C2 is comprised between the pressures p1 and p3.

49. Bioreactor according to one at least of claims 31 to 48, characterized in that the pumps P1 and P3 are stopped when the configuration of inlet and outlet of the second dynamic liquid media F2 is set in a closed loop or open loop depending upon the function which has been attributed to said liquid media.

50. Bioreactor according to claim 49, characterized in that the dynamic liquid media F2 is used to introduce into the culture chamber the cells to cultivate and to recover the cells after culture.

51. Bioreactor according to claim 49, characterized in that the dynamic liquid media F2 is used to introduce vectors of genes transfer into the culture chamber containing the cells in culture and to recover the cultivated cells, genetically modified.

52. Bioreactor according to claim 49, characterized in that the dynamic liquid media F2 is used to introduce a rinsing liquid of the molecules inhibiting the growth of the cells in culture and to remove said molecules.

53. Bioreactor according to claim 50, characterized in that when said media F2 is inoculated in the zone C2 of the cellular culture chamber through a biocompatible septum by means of a syringe, the electro-valve V2E1 is open while the electro-valves V2E2, V2E3, V2S1, V2S2 and V2S3 are closed.

54. Bioreactor according to claim 50, characterized in that for the recovery of cells after culture in the culture chamber, the electro-valve V2S1 is open, the electro-valves V2E2, V2E3, V2S2 and V2S3 are closed and the pump P2 is started in order to purge the content of the zone C2 in the cellular collecting container.

55. Bioreactor according to claim 52, characterized in that when the second media F2 contains the elements necessary for the rinsing of the cellular containment space, for the elimination of inhibiting molecules, as said media is introduced, the electro-valve V2E3 is open and the electro-valves V2E1, V2E2 are closed.

56. Bioreactor according to claim 52, characterized in that the second media F2 of rinsing flows continuously in open loop and that during the introduction and the recovery of said rinsing media, the electro-valves V2E1, V2E2, V2S1, V2S2 are closed while the electro-valves V2E3, V2S3 are open.

57. Bioreactor according to claim 51, characterized in that for the inoculation of gene transfer vectors contained in said media F2 circulating in a closed loop in the zone C2 of cells culture chamber, the electro-valve V2E2 is open, while the electro-valves V2E1, V2E3, V2S1 and V2S3 are closed.

58. Bioreactor according to one at least of claims 31 to 57, characterized in that the regulation-control unit receives the totality of the information related to the dynamic liquid media F1, F2, F3 through control and regulation means, and the information relative to the different vessels/tanks, pumps, valves and pressures prevailing in the zones C1, C2 and C3 of the culture chamber, processes said information and dispatches the necessary functioning order signals.
